(19) United States
(12) Patent Application Publication
(10) Pub. No.: US 2001/0036454 A1
(43) Pub. Date: Nov. 1, 2001

(54) Genetic modification of the lung as a portal for gene delivery

(76) Inventors: Chester Li, Acton, MA (US); Robin Ziegler, Sterling, MA (US); Seng Cheng, Wellesley, MA (US); Nelsom Yew, West Upton, MA (US)

Correspondence Address: GENZYME CORPORATION, LEGAL DEPARTMENT, 15 PLEASANT ST CONNECTOR, FRAMINGHAM, MA, 01701-9322, US

(21) Appl. No.: 09/784,563

(22) Filed: Feb. 15, 2001

Related U.S. Application Data
(60) Provisional application No. 60/183,296, filed on Feb. 17, 2000.
(60) Provisional application No. 60/207,813, filed on May 30, 2000.

(51) Int. Cl. A61K 48/00; A61L 9/04; A61K 9/00

(52) U.S. Cl. 424/93.21; 514/44; 424/43

(57) ABSTRACT

The present invention relates to methods for treatment of systemic disorders using the lung as a depot organ for transgene delivery. Transfection of the pulmonary epithelium, particularly the deep alveolar cells, or pulmonary endothelial cells, is achieved via local administration of a transgene delivery vector to the lung. The transfected cells express the transgene, and the protein thereby expressed is communicated into the circulatory system. Once entering into the circulatory system, the protein is able to achieve a systemic therapeutic effect.

### FIELD OF THE INVENTION

The present invention relates to methods for improved systemic treatment of lysosomal storage diseases, hemophilia, and other systemic medical conditions. The methods include improved methods of gene therapy in which a gene therapy vector is administered to lung tissue, including the pulmonary epithelial cells and particularly alveolar cells of the lung, where the proteins produced by such tissue are able to enter into circulation.

### BACKGROUND OF THE INVENTION

Gene therapy is now being evaluated for a number of therapeutic indications. Typically, gene therapy vectors are administered intravenously, intramuscularly or intraperitoneally. For certain pulmonary diseases, such as cystic fibrosis, it has been suggested that gene therapy vectors may be administered through the lung. However, such therapies comprise methods of local treatment, and do not involve transfection of the pulmonary epithelium, including lung alveolar tissue, nor do such therapies require that the protein produced by such gene therapy vectors enter the blood circulation and provide systemic effects in other parts of the body.

For other diseases, such as lysosomal storage diseases and hemophilia, systemic treatment will be essential in order to achieve effective therapy for such conditions. Accordingly, the present invention provides novel methods for the effective systemic treatment of systemic disorders via methods of gene therapy in which the gene therapy vectors are administered to pulmonary epithelial cells, such as the deep alveolar tissue of the lung.

### SUMMARY OF THE INVENTION

Accordingly, it is one object of the present invention to provide methods for the systemic treatment of conditions that affect cells throughout the body, particularly lysosomal storage diseases and hemophilia. The methods of the invention provide such means through the use of intranasal, pulmonary instillation and other administration of gene therapy vectors to the pulmonary endothelium or epithelium, particularly to cells of the alveoli. These cells have ready access to the body's circulatory system, and thereby factors produced by these cells may be able to enter into the bloodstream and reach affected cells throughout the body. The inventors have found, surprisingly, that intranasal, pulmonary instillation and other administration to the lung of gene therapy vectors such as adenoviral vectors, AAV vectors and non-viral vectors using cationic amphiphilic lipids, is able to achieve expression of the transfected gene product in the lung, from which it can enter circulation and reach a wide range of tissues. Thus, where a gene therapy vector which encodes the lysosomal enzymes responsible for degradation of lysosomal storage products is administered to the lung, particularly to pulmonary endothelium or epithelium, including deep alveolar cells, there is observed a reduction in the amount of lysosomal enzyme substrates that are present in a diverse range of tissues within the body. Similarly, with intranasal, pulmonary instillation or other administration of hemophilia clotting factors to the lung, it is expected that the blood clotting factor produced by the transfected pulmonary cells will be able to enter circulation and achieve therapeutic levels of enzyme in the patient's bloodstream.

The Lung and Secretion Through the Pulmonary Epithelium

The lungs are the organs that the body uses to provide oxygen to all the cells and tissues of the body. Air is drawn into the lungs through the airway, which begins at the nose and mouth. The airway is lined with hairs, called cilia, and a mucus layer, which together act to filter out dust and other particulate matter that may be in the air. Air flows across the larynx to the main airway, or the trachea. The trachea branches into the left and right lung, and each branch divides further into countless numbers of thinner passages, each ending in a cluster of air sacs, or alveoli. The alveoli are covered by a semi-permeable membrane that separates the air passage from blood vessels. It is across this membrane that oxygen moves from the airways into the blood for circulation throughout the body, at the same time, that carbon dioxide and other gases which are produced by cell metabolism move from the blood to the airways. By virtue of the irregular surfaces of the alveoli, the lungs comprise a huge area over which gases may be exchanged (and, fortuitously, for drugs to be absorbed into the bloodstream).

The pulmonary alveolar epithelium is responsible for gas exchange and oxygen transport, whereby oxygen from the air sacs of the lung is exchanged with carbon dioxide in the blood. Oxygen, once entered into the bloodstream, is circulated throughout the body. The alveolar epithelium of man contains characteristic inclusion bodies which are heterogeneous structures, but basically consist of a system of membranous profiles and a limiting membrane of the unit type. Inclusion bodies appear to result from focal cytoplasmic degradation which occurs in the rapidly changing cuboidal alveolar epithelium; however, evidence suggests that alteration of all cytoplasmic membranes may be involved in the process of inclusion body formation. There is also evidence that inclusion bodies enlarge by accretion of membranes, which finally are extruded into the alveolar space. Inclusion bodies are formed when the cuboidal alveolar epithelium is differentiating to the mature flattened type, the latter contains no inclusion bodies. On the basis of the morphologic characteristics of the inclusion bodies and the distribution of the acid phosphatase reaction, it is concluded that inclusion bodies are lysosomal structures active during remodeling of the developing alveolar epithelium. By taking advantage of the alveolar endothelial cells' access to the blood circulatory system, it is possible to efficiently achieve systemic distribution of proteins that are produced via transfection of the lung.

Lysosomal Storage Diseases

Several of the over thirty known lysosomal storage diseases (LSDs) are known to involve a similar pathogenesis, namely, a compromised lysosomal hydrolase. Generally, the activity of a single lysosomal hydrolytic enzyme is reduced or lacking altogether, usually due to inheritance of an autosomal recessive mutation. As a consequence, the substrate of the compromised enzyme accumulates undigested in lysosomes, producing severe disruption of cellular architecture and various disease manifestations.

Gaucher's disease is the oldest and most common lysosomal storage disease known. Type 1 is the most common among three recognized clinical types and follows a chronic course which does not involve the nervous system. Types 2 and 3 both have a CNS component, the former being an acute infantile form with death by age two and the latter a subacute juvenile form. The incidence of Type 1 Gaucher's disease is about one in 50,000 live births generally and about one in 400 live births among Ashkenazis (see generally Kolodny et al., 1998, “Storage Diseases of the Reticuloendothelial System”, In: Nathan and Oski's Hematology of Infancy and Childhood, 5th ed., vol. 2, David G. Nathan and Stuart H. Orkin, Eds., W.B. Saunders Co., pages 1461-1507). Also known as glucosylceramide lipidosis, Gaucher's disease is caused by inactivation of the enzyme glucocerebrosidase and accumulation of glucocerebroside.

Glucocerebrosidase normally catalyzes the hydrolysis of glucocerebroside to glucose and ceramide. In Gaucher's disease, glucocerebroside accumulates in tissue macrophages which become engorged and are typically found in liver, spleen and bone marrow and occasionally in lung, kidney and intestine. Secondary hematologic sequelae include severe anemia and thrombocytopenia in addition to the characteristic progressive hepatosplenomegaly and skeletal complications, including osteonecrosis and osteopenia with secondary pathological fractures.

Niemann-Pick disease, also known as sphingomyelin lipidosis, comprises a group of disorders characterized by foam cell infiltration of the reticuloendothelial system. Foam cells in Niemann-Pick become engorged with sphingomyelin and, to a lesser extent, other membrane lipids including cholesterol. Niemann-Pick is caused by inactivation of the enzyme acid sphingomyelinase in Types A and B disease, with 27-fold more residual enzyme activity in Type B (see Kolodny et al., 1998, Id.). The pathophysiology of major organ systems in Niemann-Pick can be briefly summarized as follows. The spleen is the most extensively involved organ of Type A and B patients. The lungs are involved to a variable extent, and lung pathology in Type B patients is the major cause of mortality due to chronic bronchopneumonia. Liver involvement is variable, but severely affected patients may have life-threatening cirrhosis, portal hypertension, and ascites. The involvement of the lymph nodes is variable depending on the severity of disease. Central nervous system (CNS) involvement differentiates the major types of Niemann-Pick. While most Type B patients do not experience CNS involvement, it is characteristic in Type A patients. The kidneys are only moderately involved in Niemann Pick disease.

Fabry disease is an X-linked recessive LSD characterized by a deficiency of α-galactosidase A (α-Gal A), also known as ceramide trihexosidase, which leads to vascular and other disease manifestations via accumulation of glycosphingolipids with terminal α-galactosyl residues, such as globotriaosylceramide (GL-3) (see generally Desnick R J et al., 1995, α-Galactosidase A Deficiency: Fabry Disease, In: The Metabolic and Molecular Bases of Inherited Disease, Scriver et al., eds., McGraw-Hill, New York, 7^{th} ed., pages 2741-2784). Symptoms may include anhidrosis (absence of sweating), painful fingers, left ventricular hypertrophy, renal manifestations, and ischemic strokes. The severity of symptoms varies dramatically (Grewal RP, 1994, Stroke in Fabry's Disease, J. Neurol. 241, 153-156). A variant with manifestations limited to the heart is recognized, and its incidence may be more prevalent than once believed (Nakao S, 1995, An Atypical Variant of Fabry's Disease in Men with Left Ventricular Hypertrophy, N. Engl. J. Med. 333, 288-293).

Recognition of unusual variants can be delayed until quite late in life, although diagnosis in childhood is possible with clinical vigilance (Ko YH et al., 1996, Atypical Fabry's Disease An Oligosymptomatic Variant, Arch. Pathol. Lab. Med. 120, 86-89; Mendez MF et al., 1997, The Vascular Dementia of Fabry's Disease, Dement. Geriatr. Cogn. Disord. 8, 252-257; Shelley ED et al., 1995, Painful Fingers, Heat Intolerance, and Telangiectases of the Ear: Easily Ignored Childhood Signs of Fabry Disease, Pediatric Derm. 12, 215-219). The mean age of diagnosis of Fabry disease is 29 years. Replacement of the defective enzyme is considered feasible using a recombinant retrovirus carrying the cDNA encoding α-Gal A to transfect skin fibroblasts obtained from Fabry patients (Medin J A et al., 1996, Correction in Trans for Fabry Disease: Expression, Secretion, and Uptake of α-Galactosidase A in Patient-Derived Cells Driven by a High-Titer Recombinant Retroviral Vector, Proc. Natl. Acad. Sci. USA 93, 7917-7922).

### METHODS OF TRANSFECTION OF PULMONARY EPITHELIAL CELLS

It has been demonstrated that nucleic acids can be delivered to the lungs by different routes, including intratracheal administration of a liquid suspension of the nucleic acids and inhalation of an aqueous aerosol mist produced by a liquid nebulizer or the use of a dry powder apparatus such as that described in U.S. Pat. No. 5,780,014, the disclosure of which is incorporated by reference. Transfer of an adenoviral vector containing the cystic fibrosis transmembrane regulator [CFTR] transgene in animal studies has been generally been accomplished by intranasal instillation (Armentano et al. J.Virol. 71:2408-2416, 1997; Kaplan et al., Human Gene Therapy 9:1469-1479, 1998), although aerosol administration by inhalation to a non-human primate resulted in the expression and delivery of the CFTR transgene (McDonald et al., Human Gene Therapy 8:411-422, 1997).

The use of a liquid nebulizer may improve transfection of the lung, is easier for patients to use, and achieves better distribution. Transgene delivery using a liquid nebulizer may be aided by the preparation of compositions which are refractory to such aggregation. For example, methods to formulate polynucleotide complexes into dry powder compositions have been described in U.S. Pat. No. 5,811,406, the disclosure of which is incorporated by reference. Such aerosolized dry powder compositions are suitable for use in the methods of the present invention in order to achieve efficient transfection of the deep lung for transgene delivery. For example, suitable compositions and methods for delivery of adenoviral vectors are described in WO 00/33886, the disclosure of which is hereby incorporated by reference.

Accordingly, the present invention provides methods for the treatment of lysosomal storage diseases, hemophilia and other systemic conditions. The methods may comprise methods of administering gene therapy vectors to the pulmonary endothelium or epithelium, particularly to the deep alveolar cells of the lung, in order to achieve transfection of these cells, where the delivered gene therapy vector can be expressed, and the protein thereby expressed, secreted or engulfed into the blood circulation. Such therapy may be suitable for the treatment of systemic disorders, such as lysosomal storage diseases and hemophilia. The methods of the present invention may be performed prior to or contemporaneously with enzyme replacement therapy for the therapeutic protein of interest, such as glucocerebrosidase or acid sphingomyelinase, under conditions suitable for the expression of said DNA molecule.

The present methods have important advantages for the treatment of lysosomal storage diseases. First, the methods of the present invention allow the persistent expression of therapeutic levels of lysosomal storage enzymes or hemophilia factors to be produced from gene therapy vectors in transfected cells of the pulmonary epithelium, particularly in the alveoli, where they can enter into the blood circulation system, to reach affected cells throughout the body. Second, the methods of the present invention may allow for more effective treatment of lysosomal storage diseases and hemophilia using gene therapy in which lower dosage regimens may conveniently be used. The gene therapy methods may also be used in conjunction with enzyme replacement therapy, or therapy with small molecules affecting the lysosomal storage disorder. The present invention may allow lower dosage regimens for therapy with enzyme replacement or small molecules, as well as breaks from treatment, or less frequent dosing.

The methods of the present invention are particularly adapted towards the treatment of lysosomal storage diseases, hemophilia, and other systemic conditions in which expression from the lung and circulation to and/or uptake in a wide variety of tissues is desired. The lysosomal storage diseases include Gaucher's disease and Niemann-Pick Disease, and other lysosomal storage disorders in which associated lysosomal enzymes are deficient. Other such lysosomal storage diseases which may be suitable for the methods of the present invention include lysosomal acid lipase (LAL) (LAL deficiency), Pompe's (alpha-glucosidase), Hurler's (alpha-L iduronidase), Fabry's (alpha-galactosidase), Hunters (MPS II) (iduronate sulfatase), Morquio Syndrome (MPS IVA) (galactosamine-6-sulfatase), MPS IVB, (beta-galactosidase) and Maroteux-Lamy C (MPS VI) (arylsulfatase B). Hemophilia is a family of hereditary diseases in which one or more proteins involved in the blood clotting cascade may be missing. Such diseases include hemophilia A, in which Factor IX is deficient, hemophilia B, in which Factor VIII is deficient, Factor VII deficiency, and von Willebrand's Disease. These conditions are also suitable for treatment by the methods of the present invention.

The preferred coding DNA sequences useful for gene therapy targeting to the lung for systemic delivery include DNA sequences which encode a therapeutic protein for which expression and entry into circulation is desired. By delivery to the lung, and particularly to the deep alveolar endothelial or epithelial cells, it is believed that more of the protein expressed by the gene therapy vector may be taken up into blood circulation, and ultimately more of the protein can be delivered to and taken up by affected tissue throughout the body. In particular, preferred coding DNA sequences include those sequences encoding, glucocerebrosidase and acid sphingomyelinase, for the treatment of patients with Gaucher's Disease [see U.S. Pat. No. 5,879,680; U.S. Pat. No. 5,236,838] and Niemann-Pick Disease [see U.S. Pat. No. 5,686,240], respectively. Other preferred coding DNA sequences include those encoding alpha-glucosidase (Pompe's Disease) [see WO00/12740], alpha-L iduronidase (Hurler's Disease) [see WO9310244A1], alpha-galactosidase (Fabry Disease) [see U.S. Pat. No. 5,401,650], iduronate sulfatase (Hunters Disease (MPS II), galactosamine-6-sulfatase (MPS IVA), beta galactosidase (MPS IVB) and arylsulfatase B (MPS VI). For methods of treating hemophilia, the preferred coding DNA sequences include sequences encoding Factor VIII [see U.S. Pat. No. 4,965,199], including B-domain deleted versions thereof [see U.S. Pat. No. 4,868,112], Factor IX [see U.S. Pat. No. 4,994,371], Factor VII and Factor VIIA [see U.S. Pat. No. 4,784,950 and U.S. Pat. No. 5,633,150], Factor V [Labrouche et al., Thrombosis Research, 87:263-267 (1997)] and Von Willebrand's Factor [see Mazzini et al., Thrombosis Research, 100:489-494 (2000); Bernardi et al., Human Molecular Genetics, 2:545-548 (1993)].

The methods of the present invention may be useful for the treatment of therapeutic disorders, including lysosomal storage diseases and hemophila. The methods of the present invention may be used in conjunction with more traditional therapies, such as enzyme-replacement therapy. Thus, for the treatment of Gaucher disease, the methods of the present invention may be used in addition to treatment with recombinantly produced glucocerobrosidase, commercially available as Cerezyme® [Genzyme Corporation, Cambridge, Mass.; also see U.S. Pat. No. 5,236,838]. For treatment of Fabry disease, the methods of the present invention may be used in addition to treatment with recombinantly produced alpha-galactosidase [see U.S. Pat. No. 5,580,757]. For treatment of hemophilia B, the methods of the present invention can be used together with administration of recombinant Factor VIII, commercially available as Recombinate® [Baxter Healthcare Corporation, Deerfield, Ill.]; Kogenate®m or ReFacto® [American Home Products Corporation, Madison, N.J.]. For treatment of hemophilia A, the methods of the present invention can be used together with administration of recombinantly produced Factor IX, commercially available as BeneFIX® [American Home Products Corporation, Madison, N.J.]. For treatment of Factor VII deficiency, or hemophilia B in patients with an antibody response to Factor VIII, the methods of the present invention may be used in conjunction with recombinantly produced Factor VII or VIIA, commercially available as NovoSeven® [Novo Nordisk Pharmaceuticals, Inc., Princeton, N.J.]. Use of the methods of the present invention may allow for the use of lower doses, or less frequent dosing, with enzyme replacement therapy.

### BRIEF DESCRIPTION OF THE FIGURES

FIG. 1: Dose Response following intranasal instillation of Ad2/CMVHIαgal complexed with DEAE/Dextran in Balb/c mice

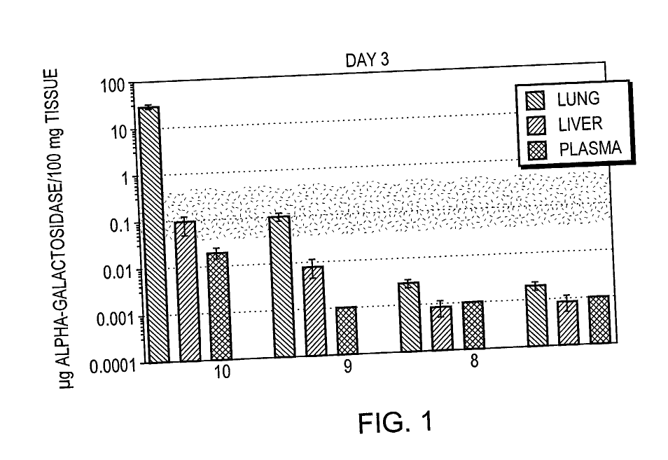

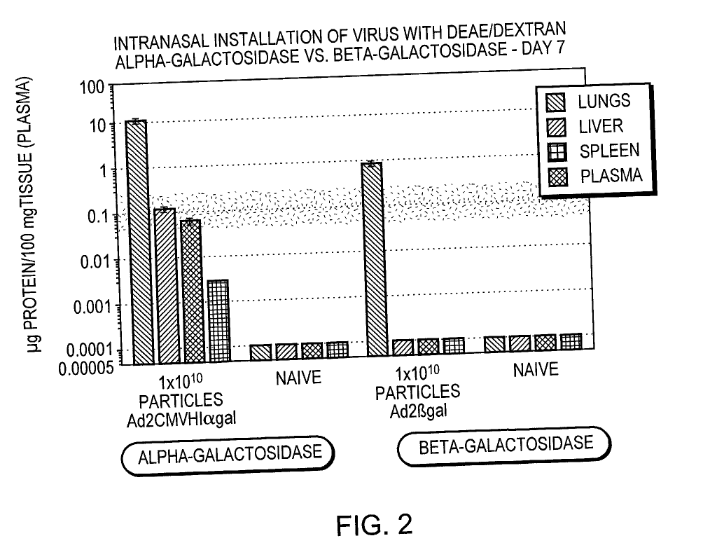

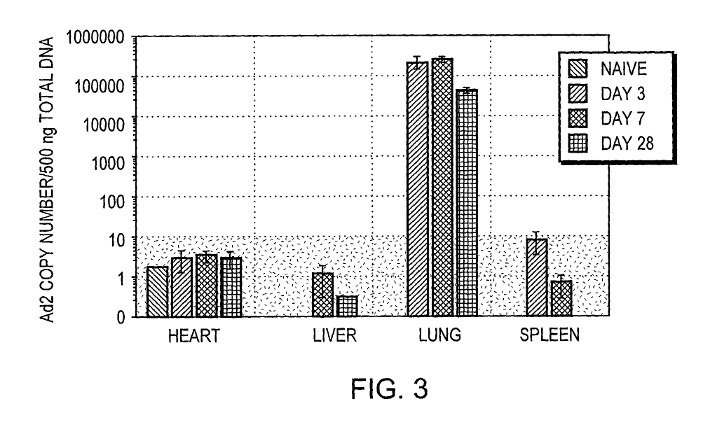

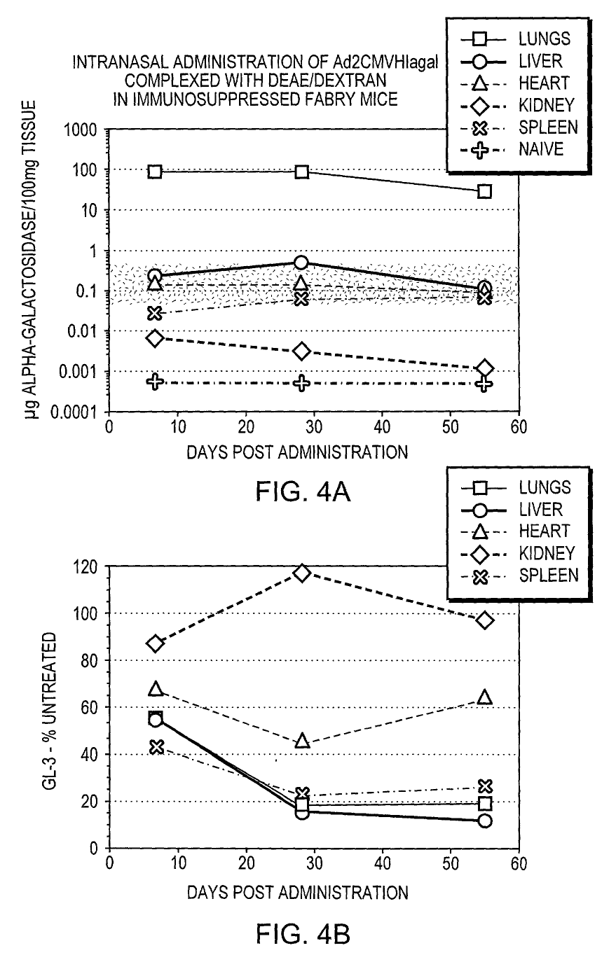

Virus was complexed with DEAE/Dextran and intranasally instilled into Balb/c mice at doses of 1×10¹⁰ particles, 1×10⁹ particles, and 1×10⁸ particles. Organs were harvested after 3 days.

Blood was collected by eyebleed at time of sacrifice. An ELISA specific for human α-galactosidase A was used to detect protein levels in tissue homogenates and plasma samples. The shaded area within the graph represents the range of α-galactosidase A in normal mouse tissues. Values represent an average of four treated mice per group. At a dose of 1×10¹⁰ particles, there were significant amounts of enzyme in the lung with levels in the liver that fall close to those in normal mice, as well as measurable enzyme in the plasma.

FIG. 2: Tissue distribution of α-galactosidase A and β-galactosidase following intranasal instillation of Ad2CMVIIIαgal vs. Ad2βgal-4

1×10¹⁰ particles of Ad2CMVHIαgal and Ad2βgal-4 were complexed with DEAE/Dextran and administered intranasally. Data shown above is from 1 week. Blood was collected by eyebleed at time of sacrifice. ELISA assays specific for human α-galactosidase A and β-galactosidase were used to detect protein levels in tissue homogenates and plasma samples. The shaded area within the graph represents the range of α-galactosidase A in normal mouse tissues. Values represent an average of four treated mice per group. Instillation of the α-galactosidase A adenovirus vector resulted in high enzyme levels in the lungs and with moderate levels in other organs such as the liver, spleen and plasma. β-galactosidase, a non-secreted protein, was limited to the lung following instillation of the β-galactosidase adenovirus vector. This suggests that the transduction is limited to the lung and that the α-galactosidase A seen outside of the lung is the result of secretion and into circulation from the lung and uptake from systemic circulation by distal tissues.

FIG. 3: Localization of viral DNA following intranasal administration

1×10¹⁰ particles of Ad2CMVHIαgal was complexed with DEAE/Dextran and administered intranasally into beige/SCID mice. Organs were harvested after 3 days, 1 week and 4 weeks. Tissues were split in half for α-galactosidase ELISA analysis and PCR quantitation. PCR analysis utilizes Taqman technology to moniter the presence of Ad2 hexon DNA. The shaded area within the graph represents values that are below the range of reliable quantitation. Values represent an average of four treated mice per group. Following intranasal administration of Ad2CMVHIαgal, the presence of Ad2 DNA appeared to be limited to the lung. This further supports the hypothesis that the transduction is limited to the lung and that the α-galactosidase A seen outside of the lung is the result of secretion and uptake.

FIG. 4: Persistence of α-galactosidase expression and reduction of GL-3 levels following intranasal administration of Ad2CMVHIαgal in immunosuppressed Fabry mice FIG. 4A.)

1×10¹⁰ particles of Ad2CMVHIαgal were complexed with DEAE/Dextran and administered intranasally into age-matched Fabry mice treated with anti-CD40 ligand, MR1. Organs were harvested 1 week, 1 month and 2 months after virus administration. The organs were divided in half for α-galactosidase and GL-3 determinations. Blood was collected by eyebleed at time of sacrifice. An ELISA specific for human α-galactosidase A was used to detect protein levels in tissue homogenates and plasma samples. The shaded area within the graph represents the range of α-galactosidase A in normal mouse tissues. Values represent an average of four treated mice per group. The α-galactosidase A levels in treated immunosuppressed Fabry mice were high in the lungs with levels in the liver and heart that fall within the range of normal animals. There were also moderate levels of enzyme measured in the spleen and detectable enzyme in the kidney. These levels persisted out to 2 months. This demonstrates that the rapid decrease of enzyme levels seen in immunocompetent mice can be averted using an immunosuppressive regimen such as MR-1.

FIG. 4B.) An ELISA-type assay based on the affinity of E. coli verotoxin to bind GL-3 was used to measure GL-3 levels in tissue extracts. Tissues were homogenized and extracted in chloroform:methanol (2:1). The neutral lipids were purified from extracts using RP-18 columns (manufactured by EM Separations). Aliquots of these extracts were dried down in Nunc Polysorp plates and analyzed for GL-3 content using porcine GL-3 (Matreya, Inc.) as a standard. Values represent an average of four treated mice per group. By 28 days after administration of virus, the levels of GL-3 in the lungs, liver, spleen and heart were significantly lower than those seen in the age-matched untreated controls. The kidney levels were not significantly reduced.

### DETAILED DESCRIPTION OF THE INVENTION

As used herein, the term “pulmonary administration” refers to administration of a formulation of the invention through the lungs by inhalation.

As used herein, the term “inhalation” refers to intake of air to the alveoli. In specific examples, intake can occur by self-administration of a formulation of the invention while inhaling, or by administration via a respirator, e.g., to a patient on a respirator. The term “inhalation” used with respect to a formulation of the invention is synonymous with “pulmonary administration.” As used herein, the term “dispersant” refers to an agent that assists aerosolization of the gene therapy vector or transfection of the lung tissue. Preferably the dispersant is pharmaceutically acceptable. As used herein, the term “pharmaceutically acceptable” means approved by a regulatory agency of the Federal or a state government as listed in the U.S. Pharmacopeia or other generally recognized pharmacopeia for use in animals, and more particularly in humans. Suitable dispersing agents are well known in the art, and include but are not limited to surfactants and the like. For example, surfactants that are generally used in the art to reduce surface induced aggregation of the protein caused by atomization of the solution forming the liquid aerosol may be used. Nonlimiting examples of such surfactants are surfactants such as polyoxyethylene fatty acid esters and alcohols, and polyoxyethylene sorbitan fatty acid esters. The surfactants and dispersants should also be chosen so as to be compatible with the gene therapy vector; e.g., substances that do not impair the infectivity of viral gene therapy vectors. Amounts of surfactants used will vary, being generally within the range or 0.001 and 4% by weight of the formulation. In a specific aspect, the surfactant is polyoxyethylene sorbitan monooleate or sorbitan trioleate. Suitable surfactants are well known in the art, and can be selected on the basis of desired properties, depending on the specific formulation, concentration of gene therapy vector, diluent (in a liquid formulation) or form of powder (in a dry powder formulation), etc.

Moreover, depending on the choice of the gene therapy vector, the desired therapeutic effect, the quality of the lung tissue (e.g., diseased or healthy lungs), and numerous other factors, the liquid or dry formulations can comprise additional components, as discussed further below.

The liquid aerosol formulations may contain the gene therapy vector and a dispersing agent in a physiologically acceptable diluent. The dry powder aerosol formulations of the present invention consist of a finely divided solid form of the gene therapy vector and a dispersing agent. With either the liquid or dry powder aerosol formulation, the formulation must be aerosolized. That is, it must be broken down into liquid or solid particles in order to ensure that the aerosolized dose actually reaches the alveoli. In general, the mass median dynamic diameter will be 5 micrometers or less, preferably less than about 2 micrometers, in order to ensure that the gene therapy vector particles reach the deep pulmonary epithelium (Wearley, L. L., 1991, 1991, Crit. Rev. in Ther. Drug Carrier Systems 8:333). The term “aerosol particle” is used herein to describe the liquid or solid particle suitable for pulmonary administration, i.e., that will reach the pulmonary epithelium, including the alveoli, or the endothelium. Other considerations such as construction of the delivery device, additional components in the formulation and particle characteristics are important. These aspects of pulmonary administration of a drug, in this case, the gene therapy vector, are well known in the art, and manipulation of formulations, aerosolization means and construction of a delivery device require at most routine experimentation by one of ordinary skill in the art.

With regard to construction of the delivery device, any form of aerosolization known in the art, including but not limited to nebulization, atomization or pump aerosolization of a liquid formulation, and aerosolization of a dry powder formulation, can be used in the practice of the invention. A delivery device that is uniquely designed for administration of solid formulations is envisioned. Often, the aerosolization of a liquid or a dry powder formulation will require a propellant. The propellant may be any propellant generally used in the art. Specific nonlimiting examples of such useful propellants are a chlorofluorocarbon, a hydrofluorocarbon, a hydochlorofluorocarbon, or a hydrocarbon, including trifluoromethane, dichlorodifluoromethane, dichlorotetrafluoroethanol, and 1,1,1,2-tetrafluoroethane, or combinations thereof.

In a preferred aspect of the invention, the device for aerosolization is a metered dose inhaler. A metered dose inhaler provides a specific dosage when administered, rather than a variable dose depending on administration. Such a metered dose inhaler can be used with either a liquid or a dry powder aerosol formulation. Metered dose inhalers are well known in the art.

Once the transgene delivery vector reaches the lung, a number of formulation-dependent factors affect the drug absorption. It will be appreciated that in treating a systemic disease or disorder that requires circulatory levels of the relevant therapeutic protein, such factors as aerosol particle size, aerosol particle shape, the presence or absence of infection, lung disease or emboli may affect the absorption of the transgene delivery vector, expression of the protein and ultimately entry of the protein into circulation. Complexing agents, such as DEAE-dextran, cationic lipids, and polycations, may be used to improve transfection efficiency. For each of the formulations described herein, certain lubricators, absorption enhancers, protein stabilizers or suspending agents may be appropriate. The choice of these additional agents will vary depending on the goal. It will be appreciated that in instances where systemic delivery of the protein is desired or sought, such as in the methods of the present invention, such variables contributing to absorption enhancement will be very important.

In a further embodiment, an aerosol formulation of the present invention can include other active ingredients in addition to the transgene delivery component. In a preferred embodiment, such active ingredients are those used for the treatment of lung disorders, and thereby may contribute to enhanced absorption of the transgene delivery vector into the pulmonary epithelium. For example, such additional active ingredients include, but are not limited to, bronchodilators, antihistamines, epinephrine, and the like, which are useful in the treatment of asthma. In another embodiment, the additional active ingredient can be an antibiotic, e.g., for the treatment of pneumonia. In a preferred embodiment, the antibiotic is tobramycin or pentamidine.

In general, the transgene delivery vector of the present invention, which encodes a protein for expression in the lung and absorption into circulation and systemic treatment of a disease or disorder may be introduced into the subject in the aerosol form in an amount designed to produce between 0.01 mg per kg body weight of the mammal up to about 100 mg per kg body weight of said mammal. One of ordinary skill in the art can readily determine a volume or weight of aerosol corresponding to this dosage based on the concentration of gene therapy vector in an aerosol formulation of the invention; alternatively, one can prepare an aerosol formulation which with the appropriate dosage of gene therapy vector in the volume to be administered, as is readily appreciated by one of ordinary skill in the art. It is also clear that the dosage will be higher in the case of inhalation therapy for a systemic disease or disorder, since therapeutic doses of the expressed protein must reach the affected tissue. It is an advantage of the present invention that administration of a transgene delivery vector directly to the lung allows use of a lower dose of enzyme replacement therapy, and may thus limit both cost and unwanted side effects. In addition, the use of the lung as a depot organ may have significant advantages compared to local administration of a transgene delivery vector to affected tissues, since many tissues are not able to efficiently take up and/or express such vectors. Another significant advantage is that delivery of the transgene to the lung may avoid potential systemic toxicity associated with administration of gene delivery vectors to other parts of the body, e.g., intramuscular, intravenous.

The formulation may be administered in a single dose or in multiple doses depending on the disease indication. It will be appreciated by one of skill in the art the exact amount of prophylactic or therapeutic formulation to be used will depend on the stage and severity of the disease, the physical condition of the subject, and a number of other factors.

Systems of aerosol delivery, such as the pressurized metered dose inhaler and the dry powder inhaler are disclosed in Newman, S. P., Aerosols and the Lung, Clarke, S. W. and Davia, D. editors, pp. 197-22 and can be used in connection with the present invention.

It is particularly contemplated that adenoviral vectors, other viral vectors such as adeno-associated vectors and retroviral or lentiviral vectors, lipid DNA complexes or liposome formulations may be especially effective for administration of the transgene delivery vector by inhalation. This is particularly so where long term administration is desired (See Wearley, 1991, Crit. Rev. in Ther. Drug Carrier Systems 8:333).

Gene Therapy Vectors

Adenoviral vectors for use to deliver transgenes to cells for applications such as in vivo gene therapy and in vitro study and/or production of the products of transgenes, commonly are derived from adenoviruses by deletion of the early region 1 (EI) genes (Berkner, K. L., Curr. Top. Micro. Immunol. 158L39-66 1992). Deletion of E1 genes renders such adenoviral vectors replication defective and significantly reduces expression of the remaining viral genes present within the vector. However, it is believed that the presence of the remaining viral genes in adenoviral vectors can be deleterious to the transfected cell for one or more of the following reasons: (1) stimulation of a cellular immune response directed against expressed viral proteins, (2) cytotoxicity of expressed viral proteins, and (3) replication of the vector genome leading to cell death.

One solution to this problem has been the creation of adenoviral vectors with deletions of various adenoviral gene sequences. In particular, pseudoadenoviral vectors (PAVs), also known as ‘gutless adenovirus’ or mini-adenoviral vectors, are adenoviral vectors derived from the genome of an adenovirus that contain minimal cis-acting nucleotide sequences required for the replication and packaging of the vector genome and which can contain one or more transgenes (See, U.S. Pat. No. 5,882,877 which covers pseudoadenoviral vectors (PAV) and methods for producing PAV, incorporated herein by reference). Such PAVs, which can accommodate up to about 36 kb of foreign nucleic acid, are advantageous because the carrying capacity of the vector is optimized, while the potential for host immune responses to the vector or the generation of replication-competent viruses is reduced. PAV vectors contain the 5′ inverted terminal repeat (ITR) and the 3′ ITR nucleotide sequences that contain the origin of replication, and the cis-acting nucleotide sequence required for packaging of the PAV genome, and can accommodate one or more transgenes with appropriate regulatory elements, e.g. promoter, enhancers, etc.

Other, partially deleted adenoviral vectors provide a partially-deleted adenoviral (termed “DeAd”) vector in which the majority of adenoviral early genes required for virus replication are deleted from the vector and placed within a producer cell chromosome under the control of a conditional promoter. The deletable adenoviral genes that are placed in the producer cell may include E1A/E1B, E2, E4 (only ORF6 and ORF6/7 need be placed into the cell), pIX and pIVa2. E3 may also be deleted from the vector, but since it is not required for vector production, it can be omitted from the producer cell. The adenoviral late genes, normally under the control of the major late promoter (MLP), are present in the vector, but the MLP may be replaced by a conditional promoter.

Conditional promoters suitable for use in DeAd vectors and producer cell lines include those with the following characteristics: low basal expression in the uninduced state, such that cytotoxic or cytostatic adenovirus genes are not expressed at levels harmful to the cell; and high level expression in the induced state, such that sufficient amounts of viral proteins are produced to support vector replication and assembly. Preferred conditional promoters suitable for use in DeAd vectors and producer cell lines include the dimerizer gene control system, based on the immunosuppressive agents FK506 and rapamycin, the ecdysone gene control system and the tetracycline gene control system. Also useful in the present invention may be the GeneSwitch™ technology [Valentis, Inc., Woodlands, Tex.] described in Abruzzese et al., Hum. Gene Ther. 1999 10:1499-507, the disclosure of which is hereby incorporated herein by reference.

The partially deleted adenoviral expression system is further described in WO99/57296, the disclosure of which is hereby incorporated by reference herein.

Adenoviral vectors, such as PAVs and DeAd vectors, have been designed to take advantage of the desirable features of adenovirus which render it a suitable vehicle for delivery of nucleic acids to recipient cells. Adenovirus is a non-enveloped, nuclear DNA virus with a genome of about 36 kb, which has been well-characterized through studies in classical genetics and molecular biology (Hurwitz, M. S., Adenoviruses Virology, 3^{rd} edition, Fields et al., eds., Raven Press, New York, 1996; Hitt, M. M. et al., Adenovirus Vectors, The Development of Human Gene Therapy, Friedman, T. ed., Cold Spring Harbor Laboratory Press, New York 1999). The viral genes are classified into early (designated E1-E4) and late (designated L1-L5) transcriptional units, referring to the generation of two temporal classes of viral proteins. The demarcation of these events is viral DNA replication. The human adenoviruses are divided into numerous serotypes (approximately 47, numbered accordingly and classified into 6 groups: A, B, C, D, E and F), based upon properties including hemaglutination of red blood cells, oncogenicity, DNA and protein amino acid compositions and homologies, and antigenic relationships.

Recombinant adenoviral vectors have several advantages for use as gene delivery vehicles, including tropism for both dividing and non-dividing cells, minimal pathogenic potential, ability to replicate to high titer for preparation of vector stocks, and the potential to carry large inserts (Berkner, K. L., Curr. Top. Micro. Immunol. 158:39-66, 1992; Jolly, D., Cancer Gene Therapy 1:51-64 1994).

PAVs have been designed to take advantage of the desirable features of adenovirus which render it a suitable vehicle for gene delivery. While adenoviral vectors can generally carry inserts of up to 8 kb in size by the deletion of regions which are dispensable for viral growth, maximal carrying capacity can be achieved with the use of adenoviral vectors containing deletions of most viral coding sequences, including PAVs. See U.S. Pat. No. 5,882,877 of Gregory et al.; Kochanek et al., Proc. Natl. Acad. Sci. USA 93:5731-5736, 1996; Parks et al., Proc. NatL. Acad. Sci. USA 93:13565-13570, 1996; Lieber et al., J. Virol. 70:8944-8960, 1996; Fisher et al., Virology 217:11-22, 1996; U.S. Pat. No. 5,670,488; PCT Publication No. WO96/33280, published Oct. 24, 1996; PCT Publication No. WO96/40955, published Dec. 19, 1996; PCT Publication No. WO97/25446, published Jul. 19, 1997; PCT Publication No. WO95/29993, published Nov. 9, 1995; PCT Publication No. WO97/00326, published Jan. 3, 1997; Morral et al., Hum. Gene Ther. 10:2709-2716, 1998.

Since PAVs are deleted for most of the adenovirus genome, production of PAVs requires the furnishing of adenovirus proteins in trans which facilitate the replication and packaging of a PAV genome into viral vector particles. Most commonly, such proteins are provided by infecting a producer cell with a helper adenovirus containing the genes encoding such proteins.

However, such helper viruses are potential sources of contamination of a PAV stock during purification and can pose potential problems when administering the PAV to an individual if the contaminating helper adenovirus can replicate and be packaged into viral particles.

It is advantageous to increase the purity of a PAV stock by reducing or eliminating any production of helper vectors which can contaminate preparation. Several strategies to reduce the production of helper vectors in the preparation of a PAV stock are disclosed in U.S. Pat. No. 5,882,877, issued Mar. 16, 1999; U.S. Pat. No. 5,670,488, issued Sep. 23, 1997 and International Patent Application No. PCT/US99/03483, incorporated herein by reference. For example, the helper vector may contain mutations in the packaging sequence of its genome to prevent its packaging, an oversized adenoviral genome which cannot be packaged due to size constraints of the virion, or a packaging signal region with binding sequences that prevent access by packaging proteins to this signal which thereby prevents production of the helper virus.

Other strategies include the design of a helper virus with a packaging signal flanked by the excision target site of a recombinase, such as the Cre-Lox system (Parks et al., Proc. Natl. Acad. Sci. USA 93:13565-13570, 1996; Hardy et al., J. Virol. 71:1842-1849, 1997, incorporated herein by reference); or the phage C31 integrase [see Calos et al., WO 00/11555]. Such helper vectors reduce the yield of wild-type levels.

Another hurdle for PAV manufacturing, aside from the problems with obtaining helper vector-free stocks, is that the production process is initiated by DNA transfections of the PAV genome and the helper genome into a suitable cell line, e.g., 293 cells. After cytopathic effects are observed in the culture indicating a successful infection, which may take up to from 2 to 6 days, the culture is harvested and is passaged onto a new culture of cells. This process is repeated for several additional passages, up to 7 times more, to obtain a modes cell lysate containing the PAV vector and any contaminating helper vector. See Parks et al., 1996, Proc. Natl. Acad. Sci. USA 93:13565-13570; Kochanek et al., 1996, Proc. Natl. Acad. Sci. USA 93:5731-5736. This lengthy process is not optimal for commercial scale manufacturing. Additionally, this process facilitates recombination and rearrangement events resulting in the propagation of PAV genomes with unwanted alterations. The use of adenoviruses for gene therapy is described, for example, in U.S. Pat. No. 5,882,877; U.S. patent, the disclosures of which are hereby incorporated herein by reference.

Adeno-associated virus (AAV) is a single-stranded human DNA parvovirus whose genome has a size of 4.6 kb. The AAV genome contains two major genes: the rep gene, which codes for the rep proteins (Rep 76, Rep 68, Rep 52, and Rep 40) and the cap gene, which codes for AAV replication, rescue, transcription and integration, while the cap proteins form the AAV viral particle. AAV derives its name from its dependence on an adenovirus or other helper virus (e.g., herpesvirus) to supply essential gene products that allow AAV to undergo a productive infection, i.e., reproduce itself in the host cell. In the absence of helper virus, AAV integrates as a provirus into the host cell's chromosome, until it is rescued by superinfection of the host cell with a helper virus, usually adenovirus (Muzyczka, Curr. Top. Micor. Immunol. 158:97-127, 1992).

Interest in AAV as a gene transfer vector results from several unique features of its biology. At both ends of the AAV genome is a nucleotide sequence known as an inverted terminal repeat (ITR), which contains the cis-acting nucleotide sequences required for virus replication, rescue, packaging and integration. The integration function of the ITR mediated by the rep protein in trans permits the AAV genome to integrate into a cellular chromosome after infection, in the absence of helper virus. This unique property of the virus has relevance to the use of AAV in gene transfer, as it allows for a integration of a recombinant AAV containing a gene of interest into the cellular genome. Therefore, stable genetic transformation, ideal for many of the goals of gene transfer, may be achieved by use of rAAV vectors. Furthermore, the site of integration for AAV is well-established and has been localized to chromosome 19 of humans (Kotin et al., Proc. Natl. Acad. Sci. 87:2211-2215, 1990). This predictability of integration site reduces the danger of random insertional events into the cellular genome that may activate or inactivate host genes or interrupt coding sequences. (Ponnazhagan et al., Hum Gene Ther. 8:275-284, 1997).

There are other advantages to the use of AAV for gene transfer. The host range of AAV is broad. Moreover, unlike retroviruses, AAV can infect both quiescent and dividing cells. In addition, AAV has not been associated with human disease, obviating many of the concerns that have been raised with retrovirus-derived gene transfer vectors.

Standard approaches to the generation of recombinant rAAV vectors have required the coordination of a series of intracellular events: transfection of the host cell with an rAAV vector genome containing a transgene of interest flanked by the AAV ITR sequences, transfection of the host cell by a plasmid encoding the genes for the AAV rep and cap proteins which are required in trans, and infection of the transfected cell with a helper virus to supply the non-AAV helper functions required in trans (Muzyczka, N., Curr. Top. Micor. Immunol. 158:97-129, 1992). The adenoviral (or other helper virus) proteins activate transcription of the AAV rep gene, and the rep proteins then activate transcription of the AAV cap genes. The cap proteins then utilize the ITR sequences to package the rAAV genome into an rAAV viral particle. Therefore, the efficiency of packaging is determined, in part, by the availability of adequate amounts of the structural proteins, as well as the accessibility of any cis-acting packaging sequences required in the rAAV vector genome.

One of the potential limitations to high level rAAV production derives from limiting quantities of the AAV helper proteins required in trans for replication and packaging of the rAAV genome. Some approaches to increasing the levels of these proteins have included placing the AAV rep gene under the control of the HIV LTR promoter to increase rep protein levels (Flotte, F. R., et al., Gene Therapy 2:29-37, 1995); the use of other heterologous promoters to increase expression of the AAV helper proteins, specifically the cap proteins (Vincent, et al., J. Virol. 71:1897-1905, 1997); and the development of cell lines that specifically express the rep proteins (Yang, Q., et al., J. Virol., 68:4847-4856, 1994).

Other approaches to improving the production of rAAV vectors include the use of helper virus induction of the AAV helper proteins (Clark, et al., Gene Therapy 3:1124-1132, 1996) and the generation of a cell line containing integrated copies of the rAAV vector and AAV helper genes so that infection by the helper virus initiates rAAV production (Clark et al., Human Gene Therapy 6:1329-1341, 1995).

rAAV vectors have been produced using replication-defective helper adenoviruses which contain the nucleotide sequences encoding the rAAV vector genome (U.S. Pat. No. 5,856,152 issued Jan. 5, 1999) or helper adenoviruses which contain the nucleotide sequences encoding the AAV helper proteins (PCT International Publication WO95/06743, published Mar. 9, 1995). Production strategies which combine high level expression of the AAV helper genes and the optimal choice of cis-acting nucleotide sequences in the rAAV vector genome have been described (PCT International Application No. WO97/09441 published Mar. 13, 1997).

Current approaches to reducing contamination of rAAV vector stocks by helper viruses, therefore, involve the use of temperature-sensitive helper viruses (Ensigner et al., J. Virol., 10:328-339, 1972), which are inactivated at the non-permissive temperature. Alternatively, the non-AAV helper genes can be subcloned into DNA plasmids which are transfected into a cell during rAAV vector production (Salvetti et al., Hum. Gene Ther. 9:695-706, 1998; Grimm, et al., Hum. Gene Ther. 9:2745-2760, 1998; WO97/09441). The use of AAV for gene therapy is described, for example, in U.S. Pat. No. 5,753,500, the disclosures of each of the above are hereby incorporated herein by reference.

Retrovirus vectors are a common tool for gene delivery (Miller, Nature (1992) 357:455-460). The ability of retrovirus vectors to deliver an unrearranged, single copy gene into a broad range of rodent, primate and human somatic cells makes retroviral vectors well suited for transferring genes to a cell.

Retroviruses are RNA viruses wherein the viral genome is RNA. When a host cell is infected with a retrovirus, the genomic RNA is reverse transcribed into a DNA intermediate which is integrated very efficiently into the chromosomal DNA of infected cells. This integrated DNA intermediate is referred to as a provirus. Transcription of the provirus and assembly into infectious virus occurs in the presence of an appropriate helper virus or in a cell line containing appropriate sequences enabling encapsidation without coincident production of a contaminating helper virus. A helper virus is not required for the production of the recombinant retrovirus if the sequences for encapsidation are provided by co-transfection with appropriate vectors.

Another useful tool for producing recombinant retroviral vectors are packaging cell lines which supply in trans the proteins necessary for producing infectious virions, but those cells are incapable of packaging endogenous viral genomic nucleic acids (Watanabe & Termin, Molec. Cell. Biol. (1983) 3(12):2241-2249; Mann et al., Cell (1983) 33:153-159; Embretson & Temin, J. Virol. (1987) 61(9):2675-2683). One approach to minimize the likelihood of generating RCR in packaging cells is to divide the packaging functions into two genomes, for example, one which expresses the gag and pol gene products and the other which expresses the env gene product (Bosselman et al., Molec. Cell. Biol. (1987) 7(5):1797-1806; Markowitz et al, J. Virol. (1988) 62(4):1120-1124; Danos &Mulligan, Proc. Natl. Acad. Sci. (1988) 85:6460-6464). That approach minimizes the ability for co-packaging and subsequent transfer of the two-genomes, as well as significantly decreasing the frequency of recombination due to the presence of three retroviral genomes in the packaging cell to produce RCR.

In the event recombinants arise, mutations (Danos & Mulligan, supra) or deletions (Boselman et al., supra; Markowitz et al., supra) can be configured within the undesired gene products to render any possible recombinants non-functional. In addition, deletion of the 3′ LTR on both packaging constructs further reduces the ability to form functional recombinants.

The retroviral genome and the proviral DNA have three genes: the gag, the pol, and the env, which are flanked by two long terminal repeat (LTR) sequences. The gag gene encodes the internal structural (matrix, capsid, and nucleocapsid) proteins; the pol gene encodes the RNA-directed DNA polymerase (reverse transcriptase) and the env gene encodes viral envelope glycoproteins. The 5′ and 3′ LTRs serve to promote transcription and polyadenylation of the virion RNAs. The LTR contains all other cis-acting sequences necessary for viral replication. Lentiviruses have additional genes including vit vpr, tat, rev, vpu, nef, and vpx (in HIV-1, HIV-2 and/or SIV). Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the result is a cis defect which prevents encapsidation of genomic RNA. However, the resulting mutant is still capable of directing the synthesis of all varion proteins.

Lentiviruses are complex retroviruses which, in addition to the common retroviral genes gag, pol and env, contain other genes with regulatory or structural function. The higher complexity enables the lentivirus to modulate the life cycle thereof, as in the course of latent infection. A typical lentivirus is the human immunodeficiency virus (HIV), the etiologic agent of AIDS. In vivo, HIV can infect terminally differentiated cells that rarely divide, such as lymphocytes and macrophages. In vitro, HIV can infect primary cultures of monocyte-derived macrophages (MDM) as well as HeLa-Cd4 or T lymphoid cells arrested in the cell cycle by treatment with aphidicolin or gamma irradiation. Infection of cells is dependent on the active nuclear import of HIV preintegration complexes through the nuclear pores of the target cells. That occurs by the interaction of multiple, partly redundant, molecular determinants in the complex with the nuclear import machinery of the target cell. Identified determinants include a functional nuclear localization signal (NLS) in the gag matrix (MA) protein, the karyophilic virion-associated protein, vpr, and a C-terminal phosphotyrosine residue in the gag MA protein. The use of retroviruses for gene therapy is described, for example, in U.S. Pat. No. 6,013,516; and U.S. Pat. No. 5,994,136, the disclosures of which are hereby incorporated herein by reference.

Other methods for delivery of transgenes to cells do not use viruses for delivery. For example, cationic amphiphilic compounds can be used to deliver the nucleic acid of the present invention. Because compounds designed to facilitate intracellular delivery of biologically active molecules must interact with both non-polar and polar environments (in or on, for example, the plasma membrane, tissue fluids, compartments within the cell, and the biologically active molecular itself), such compounds are designed typically to contain both polar and non-polar domains. Compounds having both such domains may be termed amphiphiles, and many lipids and synthetic lipids that have been disclosed for use in facilitating such intracellular delivery (whether for in vitro or in vivo application) meet this definition. One particularly important class of such amphiphiles is the cationic amphiphiles. In general, cationic amphiphiles have polar groups that are capable of being positively charged at or around physiological pH, and this property is understood in the art to be important in defining how the amphiphiles interact with the many types of biologically active (therapeutic) molecules including, for example, negatively charged polynucleotides such as DNA.

Examples of cationic amphiphilic compounds that have both polar and non-polar domains and that are stated to be useful in relation to intracellular delivery of biologically active molecules are found, for example, in the following references, which contain also useful discussion of (1) the properties of such compounds that are understood in the art as making them suitable for such applications, and (2) the nature of structures, as understood in the art, that are formed by complexing of such amphiphiles with therapeutic molecules intended for intracellular delivery.

(1) Felgner, et al., Proc. Natl. Acad. Sci. USA, 84, 7413-7417 (1987) disclose use of positively-charged synthetic cationic lipids including N→1(2,3-dioleyloxy)propyl!-N,N,N-trimethylammonium chloride (“DOTMA”), to form lipid/DNA complexes suitable for transfections. See also Felgner et al., The Journal of Biological Chemistry, 269(4), 2550-2561 (1994).

(2) Behr et al., Proc. Natl. Acad. Sci USA, 86, 6982-6986 (1989) disclose numerous amphiphiles including dioctadecylamidologlycylspermine (“DOGS”).

(3) U.S. Pat. No. 5,283,185 to Epand et al. describes additional classes and species of amphiphiles including 3.beta.>N-(N.sup.1,N.sup.1-dimethylaminoethane) carbamoyl! cholesterol, termed “DC-chol”.

(4) Additional compounds that facilitate transport of biologically active molecules into cells are disclosed in U.S. Pat. No. 5,264,618 to Felgner et al. See also Felgner et al., The Journal Of Biological Chemistry, 269(4), pp. 2550-2561 (1994) for disclosure therein of further compounds including “DMRIE” 1,2-dimyristyloxypropyl-3-dimethyl-hydroxyethyl ammonium bromide.

(5) Reference to amphiphiles suitable for intracellular delivery of biologically active molecules is also found in U.S. Pat. No. 5,334,761 to Gebeyehu et al., and in Felgner et al., Methods (Methods in Enzymology), 5, 67-75 (1993).

The use of compositions comprising cationic amphiphilic compounds for gene delivery is described, for example, in U.S. Pat. No. 5,049,386; U.S. 5,279,833; US 5,650,096; US 5,747,471; US 5,767,099; US 5,910,487; US 5,719,131; US 5,840,710; US 5,783,565; US 5,925,628; US 5,912,239; US 5,942,634; US 5,948,925; US 6,022,874;U.S. 5,994,317; U.S. 5,861,397; U.S. 5,952,916; U.S. 5,948,767; U.S. 5,939,401; and U.S. 5,935,936, the disclosures of which are hereby incorporated herein by reference.

In addition, the transgenes of the present invention can be delivered using “naked DNA”. Methods for delivering a non-infectious, non-integrating DNA sequence encoding a desired polypeptide or peptide operably linked to a promoter, free from association with transfection-facilitating proteins, viral particles, liposomal formulations, charged lipids and calcium phosphate precipitating agents are described in U.S. Pat. No. 5,580,859; U.S. 5,963,622; U.S. 5,910,488; the disclosures of which are hereby incorporated herein by reference.

Gene transfer systems that combine viral and nonviral components have also been reported. Cristiano et al., (1993) Proc. Natl. Acad. Sci. USA 90:11548; Wu et al. (1994) J. Biol. Chem. 269:11542; Wagner et al. (1992) Proc. Natl. Acad. Sci. USA 89:6099; Yoshimura et al. (1993) J. Biol. Chem. 268:2300; Curiel et al. (1991) Proc. Natl. Acad. Sci. USA 88:8850; Kupfer et al. (1994) Human Gene Ther. 5:1437; and Gottschalk et al.(1994) Gene Ther. 1:185. Inmost cases, adenovirus has been incorporated into the gene delivery systems to take advantage of its endosomolytic properties. The reported combinations of viral and nonviral components generally involve either covalent attachment of the adenovirus to a gene delivery complex or cointernalization of unbound adenovirus with cationic lipid: DNA complexes.

Aerosol Dry Powder Formulations

It is also contemplated that the present pharmaceutical formulation will be used as a dry powder inhaler formulation comprising a finely divided powder form comprising the transgene delivery vector and a optionally a dispersant. The form of the transgene delivery vector will generally be a lyophilized powder. Lyophilized forms of transgene delivery vector can be obtained through standard techniques.

In another embodiment, the dry powder formulation will comprise a finely divided dry powder containing one or more transgene delivery vectors, a dispersing agent and also a bulking agent. Bulking agents useful in conjunction with the present formulation include such agents as lactose, sorbitol, sucrose, or mannitol, in amounts that facilitate the dispersal of the powder from the device.

What constitutes a therapeutically effective amount in a particular case will depend on a variety of factors within the knowledge of the skilled practitioner. Such factors include the physical condition of the subject being treated, the severity of the condition being treated, the disorder or disease being treated, and so forth. In general, any statistically significant attenuation of one or more symptoms associated with the systemic disease or disorder constitutes treatment within the scope of the present invention. It is anticipated that for most mammals, including humans, the administered dose for pulmonary delivery of gene therapy vectors should be targeted for the delivery of adenoviral or AAV particles, generally in the range of about 10⁶ to about 10¹⁵ particles, more preferably in the range of about 10⁸ to about 10¹³ particles. In the particular embodiments wherein retroviral or lentiviral vectors are used, the dose of the DNA encoding modified FVII can be delivered via retroviral or lentiviral particles, generally in the range of about 10⁴ to about 10¹³ particles, more preferably in the range of about 10⁶ to about 10¹¹ particles. When the transgene is delivered in the form of plasmid DNA, a useful dose will generally range from about 1 ug to about 1 g of DNA, preferably in the range from about 100 ug to about 100 mg of DNA. The above can be modified to effect entry into systemic circulation of the therapeutic protein expressed from said transgene in an amount ranging from about 0.01 mg/kg to 100 mg/kg body weight of the patient.

It is contemplated that transgene delivery vectors, or more preferably the formulations of the present invention, can be administered to a subject in need of prophylactic or therapeutic treatment. As used herein, the term “subject” refers to an animal, more preferably a mammal, and most preferably a human.

It is envisioned that the transgene delivery vectors will be delivered to achieve elevation of plasma levels of the protein expressed from the transgene, to treat diseases or disorders that involve a deficiency of a naturally occurring factor, such as a lysosomal enzyme or a blood clotting factor. Diseases or disorders that require systemic or circulating levels of a therapeutic protein, and thus suitable for treatment by the methods of the present invention are detailed above, and include lysosomal storage enzymes and blood clotting factors.

Aerosol administration is an effective means for delivering the transgene delivery vectors of the invention directly to the respiratory tract, particularly the alveoli. Some of the advantages of this method are: 1) it circumvents the effects of enzymatic degradation, poor absorption from the gastrointestinal tract, or loss of the therapeutic agent due to the hepatic first-pass effect; 2) it administers active ingredients which would otherwise fail to reach their target sites in the respiratory tract due to their molecular size, charge or affinity to extra-pulmonary sites; 3) it provides for fast absorption into the body via the alveoli of the lungs; and 4) it avoids exposing other organ systems to the active ingredient, which is important where exposure might cause undesirable side effects. For these reasons, aerosol administration is particularly advantageous for treatment of diseases or disease conditions involving systemic disorders.

There are three types of pharmaceutical inhalation devices most heavily used: nebulizer inhalers, metered-dose inhalers and dry powder inhalers. Nebulizer devices produce a stream of high velocity air that causes the transgene delivery vector (which has been formulated in a liquid form) to spray as a mist which is carried into the patient's respiratory tract. Metered-dose inhalers typically have the formulation packaged with a compressed gas and, upon actuation, discharge a measured amount of the transgene delivery vector by compressed gas, thus affording a reliable method of administering a set amount of agent. Dry powder inhalers administer the transgene delivery vector in the form of a free flowing powder that can be dispersed in the patient's air-stream during breathing by the device. In order to achieve a free flowing powder, the transgene delivery vector may be formulated with an excipient, such as lactose. A measured amount of the transgene delivery vector is stored in a capsule form and is dispensed to the patient with each actuation. All of the above methods can be used for administering the present invention.

Formulations of the invention can include liposomes containing a transgene delivery vector, which may be administered in combination with an amount of alveolar surfactant protein effective to enhance the transport of the protein expressed from the transgene across the pulmonary surface and into the circulatory system of the patient. Such liposomes and formulations containing such are disclosed within U.S. Pat. No. 5,006,343, issued Apr. 9, 1991, which is incorporated herein by reference to disclose liposomes and formulations of liposomes used in intrapulmonary delivery. The formulations and methodology disclosed in U.S. Pat. No. 5,006,343 can be adapted for the application of transgene delivery vectors and included within the delivery device of the present invention in order to provide for effective treatments of patients with systemic disorders.

The preferred coding DNA sequences contained in the transgene delivery vector include any therapeutic protein. In preferred embodiments, the coding DNA sequences comprise a sequence encoding a protein which is desired to be targeted systemically. In particular, preferred coding DNA sequences include those sequences encoding glucocerebrosidase for the treatment of patients with Gaucher's Disease and acid sphingomyelinase for the treatment of patients with Niemann-Pick Disease, respectively. Other preferred coding DNA sequences include those encoding alpha-glucosidase (Pompe's Disease), alpha-L iduronidase (Hurler's Disease), alphagalactosidase (Fabry's Disease), and iduronate sulfatase (Hunters Disease (MPS II), galactosamine-6-sulfatase (MPS IVA); beta-D-galactosidase (MPS IVB); and arylsulfatase B (MPS VI); Factor VIII [see U.S. Pat. No. 4,965,199], including B-domain deleted versions thereof [see U.S. Pat. No. 4,868,112], Factor IX [see U.S. Pat. No. 4,994,371], Factor VII and Factor VIIA [see U.S. Pat. No. 4,784,950 and U.S. Pat. No. 5,633,150], Factor V [Labrouche et al., Thrombosis Research, 87:263-267 (1997)] and Von Willebrand's Factor [see Mazzini et al., Thrombosis Research, 100:489-494 (2000); Bernardi et al., Human Molecular Genetics, 2:545-548 (1993)].

Methods for the purification of recombinant human proteins are well-known, including methods for the production of recombinant human glucocerebrosidase [for Gaucher's Disease]; acid sphingomyelinase [for Niemann-Pick Disease], alpha-galactosidase [for Fabry Disease]; alpha-glucosidase [for Pompe's Disease]; alpha-L iduronidase [for Hurler's Syndrome]; iduronate sulfatase [for Hunter's Syndrome]; galactosamine-6-sulfatase [for MPS IVA]; beta-D-galactosidase [for MPS IVB]; and arylsulfatase B [for MPS VI]. See, for example, Scriver et al., eds., The Metabolic and Molecular Bases of Inherited Diseases, Vol. II., 7^{th} ed. (McGraw-Hill, NY; 1995), the disclosure of which is hereby incorporated herein by reference.

As demonstrated by the experiments represented by the figures, localized and selective transduction of the lung was achieved in accordance with the methods of the present invention. At the same time, enzymatic activity was observed outside of the lung, suggeting that the enzyme crossed the air-blood barrier, entered systemic circulation and was internalized by distal tissues. The levels of enzyme activity detected in these tissues, while lower than that observed following systemic delivery of the virus, were nevetheless within the therapeutic range.

The examples, results and figures above illustrate practice of embodiments of the invention, with respect to the use of adenoviral transgene delivery vectors to the lung for the treatment of lysosomal storage diseases. The examples are not limiting in any respect, and the skilled artisan will recognize many advantageous aspects of the above disclosure, and will readily appreciate that many variations, additions and modifications to the above, including the use of other transgene delivery systems, such as lipid:DNA complexes, and reagents, are available. Such variations, additions and modifications constitute part of the present invention.

The disclosure of all of the publications cited within are hereby incorporated by reference.

We claim:

1. A method for treatment of a patient suffering from a systemic disorder or disease, comprising administering to the lung a transgene delivery vector, said transgene delivery vector comprising a nucleotide sequence which encodes for a therapeutic protein, such that the transgene delivery vector transfects lung cells, expresses the therapeutic protein, and the therapeutic protein enters into the patient's circulatory system.

2. The method of claim 1, wherein the systemic disorder or disease is a lysosomal storage disease.

3. The method of claim 1, wherein the patient is suffering from Gaucher's Disease, and the transgene delivery vector comprises a nucleotide sequence encoding glucocerebrosidase.

4. The method of claim 1, wherein the patient is suffering from Niemann-Pick Disease, and the transgene delivery vector comprises a nucleotide sequence encoding acid sphingomyelinase.

5. The method of claim 1, wherein the patient is suffering from Fabry Disease, and the transgene delivery vector comprises a nucleotide sequence encoding alpha-galactosidase.

6. The method of claim 1, wherein the patient is suffering from Pompe's Disease, and the transgene delivery vector comprises a nucleotide sequence encoding alpha glucosidase.

7. The method of claim 1, wherein the patient is suffering from Hurler's Disease, and the transgene delivery vector comprises a nucleotide sequence encoding alpha-L-iduronidase.

8. The method of claim 1, wherein the patient is suffering from Hunter's Disease, and the transgene delivery vector comprises a nucleotide sequence encoding iduronate sulfatase.

9. The method of claim 1, wherein the patient is suffering from Morquio Syndrome, and the transgene delivery vector comprises a nucleotide sequence encoding galactosamine-6-sulfatase.

10. The method of claim 1, wherein the patient is suffering from Maroteux-Lamy Disease, and the transgene delivery vector comprises a nucleotide sequence encoding arylsulfatase B.

11. The method of claim 1, wherein the systemic disorder or disease is a blood clotting deficiency.

12. The method of claim 1, wherein the patient is suffering from hemophilia A, and the transgene delivery vector comprises a nucleotide sequence encoding Factor IX.

13. The method of claim 1, wherein the patient is suffering from hemophilia B, and the transgene delivery vector comprises a nucleotide sequence encoding Factor VIII.

14. The method of claim 1, wherein the patient is suffering from hemophilia B, and the transgene delivery vector comprises a nucleotide sequence encoding Factor VIIA.

15. The method of claim 1, wherein the patient is suffering from von Willebrand's Disease, and the transgene delivery vector comprises a nucleotide sequence encoding von Willebrand's Factor.
